(19) United States
(12) Patent Application Publication
(10) Pub. No.: US 2023/0227413 A1
(43) Pub. Date: Jul. 20, 2023

(54) SOLID STATE FORMS OF ENSARTINIB AND ENSARTINIB SALTS

(71) Applicant: TEVA PHARMACEUTICALS INTERNATIONAL GMBH, Jona (CH)

(72) Inventors: Petar Bibulic, Zagreb (HR); Sanja Matecic Musanic, Zagreb (HR); Lorena Kordic, Rab (HR)

(21) Appl. No.: 18/007,821

(22) PCT Filed: Aug. 3, 2021
(86) PCT No.: PCT/US2021/044249

§ 371 Date: Dec. 2, 2022

Related U.S. Application Data
(60) Provisional application No. 63/157,909, filed on Mar. 8, 2021.
(60) Provisional application No. 63/133,514, filed on Jan. 4, 2021.
(60) Provisional application No. 63/092,270, filed on Oct. 15, 2020.
(60) Provisional application No. 63/060,186, filed on Aug. 3, 2020.

(51) Int. Cl. C07D 237/22

(52) U.S. Cl. CPC C07D 237/22; C07B 2200/13

(57) ABSTRACT

The present disclosure encompasses solid state forms and co-crystals of Ensartinib and of Ensartinib salts, in embodiments crystalline polymorphs of Ensartinib and of Ensartinib salts and co-crystals, processes for preparation thereof, and pharmaceutical compositions thereof.

### FIELD OF THE DISCLOSURE

The present disclosure encompasses solid state forms and co-crystals of Ensartinib and of Ensartinib salts, in embodiments crystalline polymorphs of Ensartinib and of Ensartinib salts and co-crystals, processes for preparation thereof, and pharmaceutical compositions thereof.

### BACKGROUND OF THE DISCLOSURE

Ensartinib, has the following chemical structure:

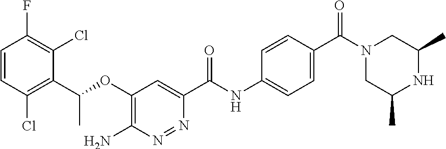 [SMILES: C[C@@H]1CN(C(=O)c2ccc(NC(=O)c3cc(O[C@H](C)c4c(Cl)ccc(F)c4Cl)c(N)nn3)cc2)C[C@H](C)N1]

Ensartinib is an orally available small molecule that is developed for the treatment of anaplastic lymphoma kinase (ALK)-positive non-small cell lung cancer (NSCLC). Ensartinib is also under investigation for the treatment of melanoma with ALK alterations or aberrant ALK expression; or, in pediatric patients, for the treatment of: recurrent, refractory or advanced solid tumors, non-Hodgkin lymphoma, or histiocytic disorders with ALK or ROS1 genomic alterations.

The compound is described in U.S. Pat. No. 9,126,947.

U.S. Patent Publication No. 2019/0135792 describes amorphous and crystalline forms of Ensartinib diHCl.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Ensartinib and of Ensartinib salts.

### SUMMARY OF THE DISCLOSURE

The present disclosure provides crystalline polymorphs and co-crystals of Ensartinib and of Ensartinib salts, processes for preparation thereof, and pharmaceutical compositions thereof. These crystalline polymorphs can be used to prepare other solid state forms of Ensartinib, Ensartinib salts and their solid state forms.

The present disclosure also provides uses of the said solid state forms and co-crystals of Ensartinib and of Ensartinib salts in the preparation of other solid state forms of Ensartinib or salts thereof.

The present disclosure provides crystalline polymorphs and co-crystals of Ensartinib and of Ensartinib salts for use in medicine, including for the treatment of cancer, in particular non-small cell lung cancer (NSCL), or for the treatment of melanoma with ALK alterations or aberrant ALK expression; or, in pediatric patients, for the treatment of: recurrent, refractory or advanced solid tumors, non-Hodgkin lymphoma, or histiocytic disorders with ALK or ROS1 genomic alterations. Preferably, the present disclosure provides crystalline polymorphs and co-crystals of Ensartinib and of Ensartinib salts and the pharmaceutical compositions and/or formulations of Ensartinib and of Ensartinib salts of the present disclosure may be used in the treatment of cancer, in particular non-small cell lung cancer (NSCLC).

The present disclosure also encompasses the use of crystalline polymorphs and co-crystals of Ensartinib and of Ensartinib salts of the present disclosure for the preparation of pharmaceutical compositions and/or formulations.

In another aspect, the present disclosure provides pharmaceutical compositions comprising crystalline polymorphs and co-crystals of Ensartinib and/or of Ensartinib salts according to the present disclosure.

The present disclosure includes processes for preparing the above mentioned pharmaceutical compositions. The processes include combining any one or a combination of the crystalline polymorphs or co-crystals of Ensartinib and/or of Ensartinib salts with at least one pharmaceutically acceptable excipient.

The crystalline polymorph or co-crystals of Ensartinib and of Ensartinib salts as defined herein and the pharmaceutical compositions or formulations of the crystalline polymorph of Ensartinib and of Ensartinib salts may be used as medicaments, such as for the treatment of cancer, or for the treatment of melanoma with ALK alterations or aberrant ALK expression; or, in pediatric patients, for the treatment of: recurrent, refractory or advanced solid tumors, non-Hodgkin lymphoma, or histiocytic disorders with ALK or ROS1 genomic alterations. Preferably, the crystalline polymorphs and co-crystals of Ensartinib and of Ensartinib salts and the pharmaceutical compositions and/or formulations of Ensartinib and of Ensartinib salts of the present disclosure may be used in the treatment of cancer, in particular non-small cell lung cancer (NSCLC).

The present disclosure also provides methods of treating cancer by administering a therapeutically effective amount of any one or a combination of the crystalline polymorphs or co-crystals of Ensartinib and/or of Ensartinib salts of the present disclosure, or at least one of the above pharmaceutical compositions, to a subject suffering from cancer, or otherwise in need of the treatment. Particularly, the present disclosure also provides methods for treating cancer, in particular non-small cell lung cancer (NSCLC), or melanoma with ALK alterations or aberrant ALK expression; or, in pediatric patients, for the treatment of: recurrent, refractory or advanced solid tumors, non-Hodgkin lymphoma, or histiocytic disorders with ALK or ROS1 genomic alterations, preferably for the treatment of cancer, in particular non-small cell lung cancer (NSCLC) by administering a therapeutically effective amount of any one or a combination of the crystalline polymorphs or co-crystals of Ensartinib and/or of Ensartinib salts of the present disclosure, or at least one of the above pharmaceutical compositions to a subject in need of the treatment.

The present disclosure also provides uses of crystalline polymorphs and co-crystals of Ensartinib and of Ensartinib salts of the present disclosure, or at least one of the above pharmaceutical compositions, for the manufacture of medicaments for treating, e.g., cancer.

### BRIEF DESCRIPTION OF THE DRAWINGS

FIG. 1 shows a characteristic X-ray powder diffraction pattern (XRPD) of Ensartinib Form 1.

FIG. 2 shows a characteristic XRPD of Ensartinib Form 2.

FIG. 3 shows a characteristic XRPD of Ensartinib hydrochloride salt Form T1.

FIG. 4 shows a characteristic XRPD of crystalline Form M1 of Ensartinib dihydrochloride: maleic acid.

FIG. 5 shows a characteristic XRPD of crystalline Form M2 of Ensartinib dihydrochloride: L-malic acid.

FIG. 6 shows a characteristic XRPD of crystalline Form M5 of Ensartinib dihydrochloride: L-tartaric acid.

FIG. 7 shows a characteristic XRPD of crystalline Form M4 of Ensartinib dihydrochloride: Succinic acid.

FIG. 8 shows a characteristic solid state ¹³C NMR spectrum of crystalline Ensartinib Form 1.

FIG. 9 shows a characteristic FT-IR spectrum of crystalline Ensartinib Form 1.

FIG. 10 shows a characteristic solid state ¹³C NMR spectrum of crystalline Ensartinib hydrochloride salt Form T1.

FIG. 11 shows a characteristic FT-IR spectrum of crystalline Ensartinib hydrochloride salt Form T1.

FIG. 12 shows a characteristic solid state ¹³C NMR spectrum of crystalline Form M1 of Ensartinib dihydrochloride: maleic acid.

FIG. 13 shows a characteristic FT-IR spectrum of crystalline Form M1 of Ensartinib dihydrochloride: maleic acid.

FIG. 14 shows a characteristic FT-IR spectrum of crystalline Form M4 of Ensartinib dihydrochloride: Succinic acid.

FIG. 15 shows a characteristic solid state ¹³C NMR spectrum of crystalline Form M5 of Ensartinib dihydrochloride: L-tartaric acid.

FIG. 16 shows a characteristic FT-IR spectrum of crystalline Form M5 of Ensartinib dihydrochloride: L-tartaric acid.

### DETAILED DESCRIPTION OF THE DISCLOSURE

The present disclosure encompasses solid state forms and co-crystals of Ensartinib and of Ensartinib salts, including crystalline polymorphs and co-crystals of Ensartinib and of Ensartinib salts, processes for preparation thereof, and pharmaceutical compositions thereof.

Solid state properties of Ensartinib and of Ensartinib salt and crystalline polymorphs thereof can be influenced by controlling the conditions under which Ensartinib and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Ensartinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Ensartinib. In some embodiments of the disclosure, the described crystalline polymorph of Ensartinib may contain from about 1% to about 20% (w/w), from about 5% to about 20% (w/w), or from about 5% to about 10% (w/w) of one or more other crystalline polymorph of the same Ensartinib.

Depending on which other crystalline polymorphs a comparison is made, the crystalline polymorphs of Ensartinib and of Ensartinib salts of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Ensartinib referred to herein as being characterized by graphical data “as depicted in” or “as substantially depicted in” a Figure will thus be understood to include any crystal forms of Ensartinib characterized with the graphical data having such small variations, as are well known to the skilled person, in comparison with the Figure.

As used herein, and unless stated otherwise, the term “anhydrous” in relation to crystalline forms of Ensartinib, relates to a crystalline form of Ensartinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “co-crystal” as used herein is defined as a crystalline material including two or more molecules in the same crystalline lattice and associated by non-ionic and non-covalent bonds. In some embodiments, the co-crystal includes two molecules which are in natural state. In embodiments the molar ratio between the active pharmaceutical ingredient (Ensartinib or Ensartinib diHCl) and the coformer (i.e. organic acid) is between 2:1 and 1:2, in embodiments 1: 1.5 and 1.5:1, in some embodiments between 1:1.25 and 1.25:1, in other embodiments about 1:1.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

As used herein, unless stated otherwise, ¹³C NMR spectra employing cross-polarization were acquired using the standard pulse scheme at magic angle spinning frequency of 11 kHz, preferably at a temperature of 293 K±3K.

As used herein, unless stated otherwise, solid state FT-IR reported herein are measured at 293 K±3K. Preferably, the solid state FT-IR is measured in a spectral range of 4000-400 cm⁻¹.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

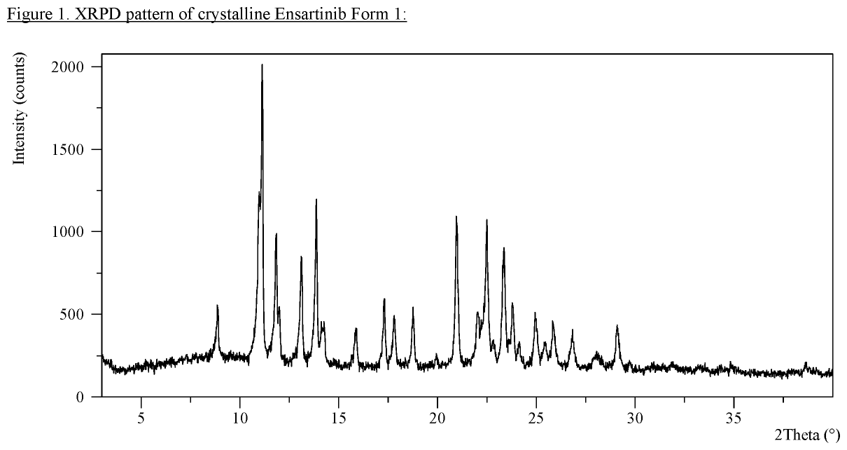

The present disclosure includes a crystalline polymorph of Ensartinib, designated Form 1. The crystalline Form 1 of Ensartinib may be characterized by data selected from one or more of the following: an X-ray powder diffraction pattern substantially as depicted in FIG. 1; an X-ray powder diffraction pattern having peaks at 11.1, 11.8, 13.9, 21.0 and 22.5 degrees 2-theta±0.2 degrees 2-theta; and combinations of these data.

According to any aspect or embodiment of the present disclosure, crystalline Form 1 of Ensartinib may be further characterized by: an X-ray powder diffraction pattern having peaks at 11.1, 11.8, 13.9, 21.0 and 22.5 degrees 2-theta±0.2 degrees 2-theta, and also having any one, two, three, four or five additional peaks selected from 8.9, 13.1, 17.3, 18.8 and 23.4 degrees 2-theta±0.2 degrees 2-theta; or an X-ray powder diffraction pattern having peaks at 8.9, 11.1, 11.8, 13.1, 13.9, 17.3, 18.8, 21.0, 22.5, and 23.4 degrees 2-theta±0.2 degrees 2-theta.

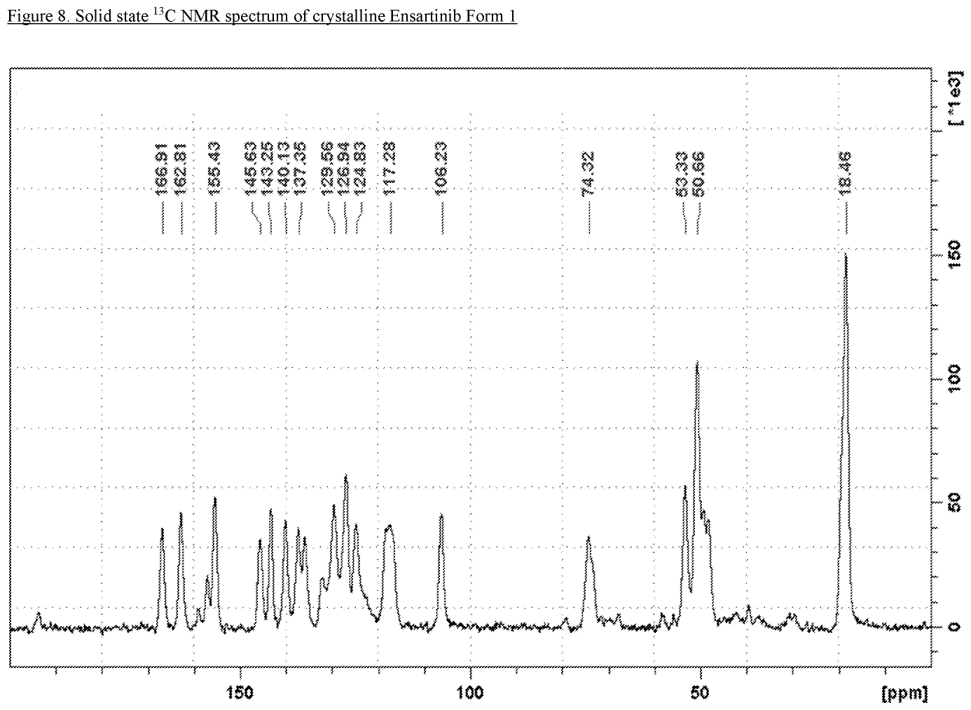

Alternatively or additionally, according to any aspect or embodiment of the present disclosure, crystalline Form 1 of Ensartinib may be characterized by data selected from one or more of the following: a solid state ¹³C NMR having peaks at 166.9, 162.8, 155.4, 143.3, 126.9, ±0.2 ppm; or a solid state ¹³C NMR spectrum having chemical shift differences between a reference peak at 106.2±0.2 ppm of: 60.7, 56.6, 49.2, 37.0, 20.7±0.1 ppm respectively; or by a solid state ¹³C NMR spectrum substantially as depicted in FIG. 8; and combinations of these data.

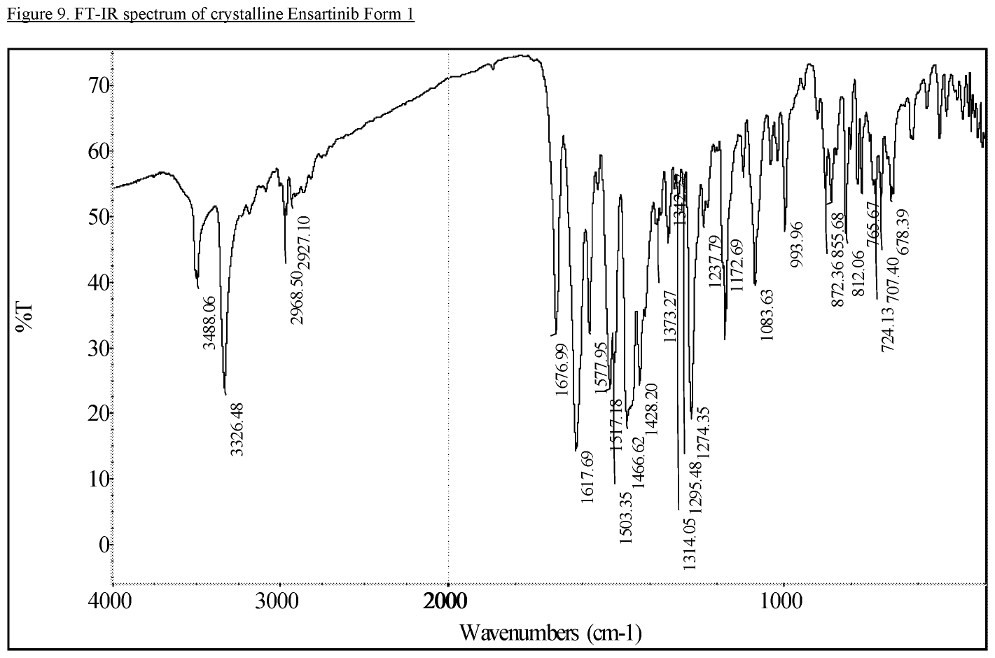

Crystalline Form 1 of Ensartinib according to any aspect or embodiment of the present disclosure may be further characterized by a FT-IR spectrum substantially as depicted in FIG. 9.

According to any aspect or embodiment of the present disclosure, crystalline Form 1 of Ensartinib is isolated.

According to any aspect or embodiment of the present disclosure, crystalline Form 1 of Ensartinib may be an anhydrous form.

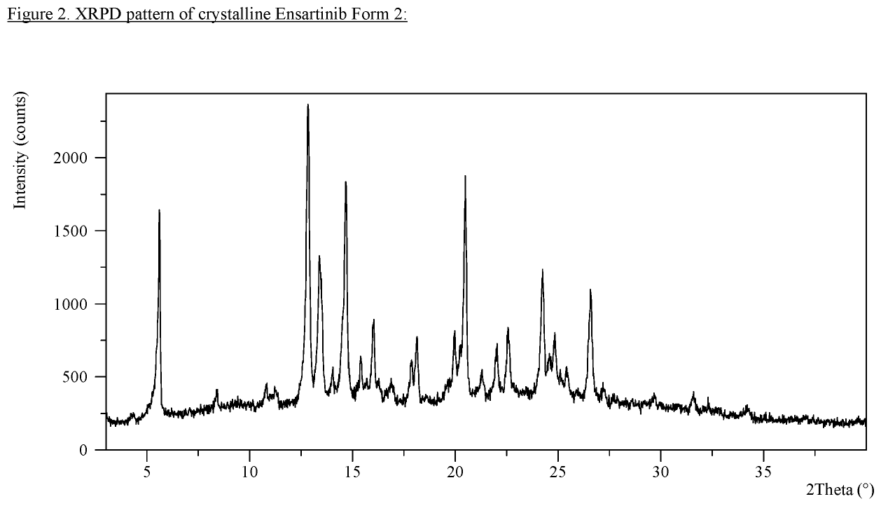

In another embodiment, the present disclosure includes a crystalline polymorph of Ensartinib, designated Form 2. The crystalline Form 2 of Ensartinib may be characterized by data selected from one or more of the following: an X-ray powder diffraction pattern substantially as depicted in FIG. 2; an X-ray powder diffraction pattern having peaks at 5.6, 12.8, 13.4, 14.7 and 20.5 degrees 2-theta±0.2 degrees 2-theta; and combinations of these data.

According to any aspect or embodiment of the present disclosure, crystalline Form 2 of Ensartinib may be further characterized by: an X-ray powder diffraction pattern having peaks at 5.6, 12.8, 13.4, 14.7 and 20.5 degrees 2-theta±0.2 degrees 2-theta, and also having any one, two, three, four or five additional peaks selected from 16.0, 18.1, 22.0, 24.2 and 26.6 degrees 2-theta±0.2 degrees 2-theta; or an X-ray powder diffraction pattern having peaks at 5.6, 12.8, 13.4, 14.7, 16.0, 18.1, 20.5, 22.0, 24.2, and 26.6. degrees 2-theta±0.2 degrees 2-theta.

According to any aspect or embodiment of the present disclosure, crystalline Form 2 of Ensartinib is isolated.

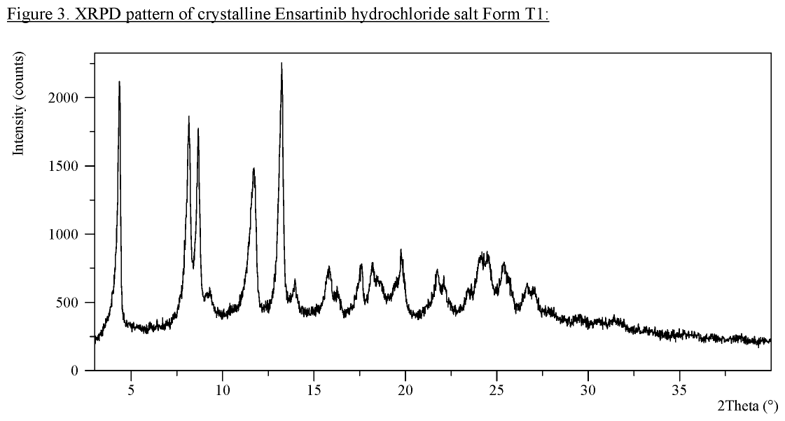

In a further embodiment, the present disclosure includes a crystalline polymorph of Ensartinib hydrochloride salt designated Form T1. The crystalline Form T1 of Ensartinib hydrochloride salt may be characterized by data selected from one or more of the following: an X-ray powder diffraction pattern substantially as depicted in FIG. 3; an X-ray powder diffraction pattern having peaks at 4.4, 8.2, 8.7, 11.7 and 13.3 degrees 2-theta±0.2 degrees 2-theta; and combinations of these data.

According to any aspect or embodiment of the present disclosure, crystalline Form T1 of Ensartinib hydrochloride salt may be further characterized by: an X-ray powder diffraction pattern having peaks at 4.4, 8.2, 8.7, 11.7 and 13.3 degrees 2-theta±0.2 degrees 2-theta, and also having any one, two, three, four or five additional peaks selected from 14.0, 15.9, 17.6, 18.2 and 19.8 degrees 2-theta±0.2 degrees 2-theta; or an X-ray powder diffraction pattern having peaks at 4.4, 8.2, 8.7, 11.7, 13.3, 14.0, 15.9, 17.6, 18.2, and 19.8 degrees 2-theta±0.2 degrees 2-theta.

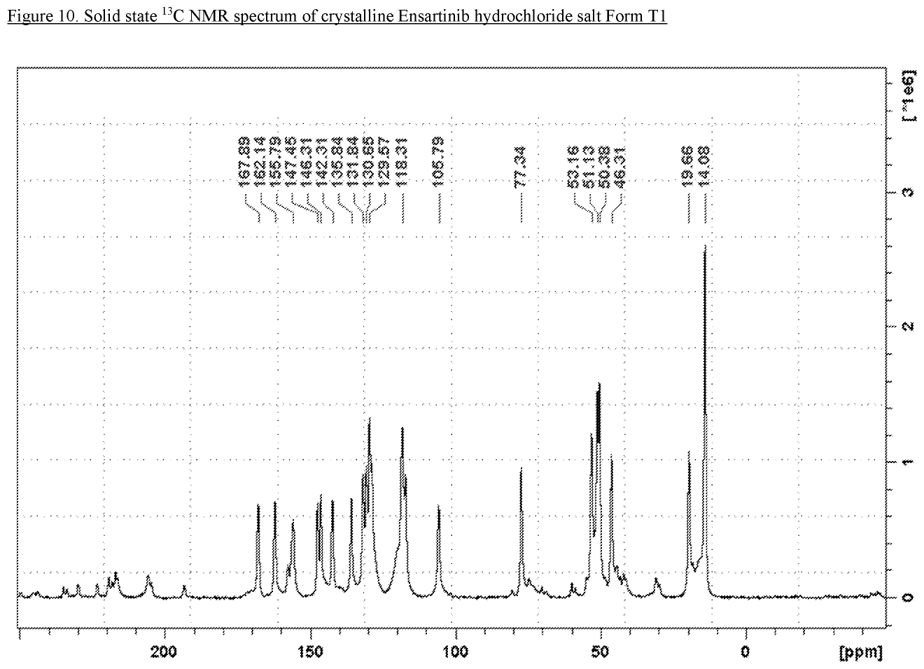

Alternatively, or additionally, according to any aspect or embodiment of the present disclosure, crystalline Form T1 of Ensartinib hydrochloride salt may be characterized by data selected from one or more of the following: a solid state ¹³C NMR having peaks at 167.9, 142.3, 130.7, 118.3, 105.8, +0.2 ppm; or a solid state ¹³C NMR spectrum having chemical shift differences between a reference peak at 77.3±0.2 ppm of: 90.6, 65.0, 53.4, 41.0, 28.5±0.1 ppm respectively; or by a solid state ¹³C NMR spectrum substantially as depicted in FIG. 10; and combinations of these data.

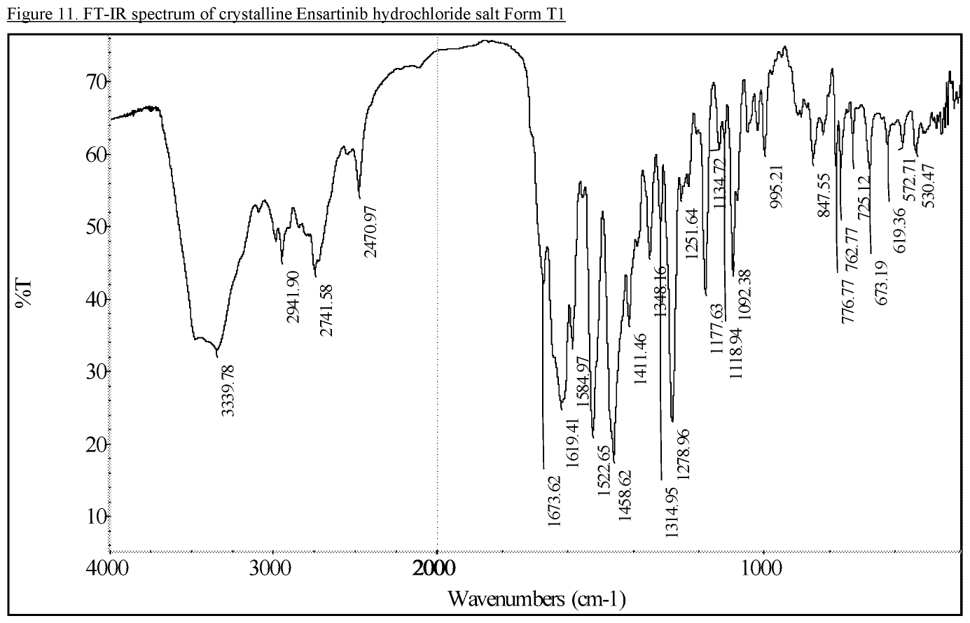

Crystalline Form T1 of Ensartinib hydrochloride according to any aspect or embodiment of the present disclosure may be further characterized by a FT-IR spectrum substantially as depicted in FIG. 11.

According to any aspect or embodiment of the present disclosure, Form T1 is the mono hydrochloride salt of Ensartinib. Preferably, according to any aspect or embodiment of the present invention, Form T1 is a monohydrochloride salt of Ensartinib.

According to any aspect or embodiment of the present disclosure, Form T1 is a hydrate, preferably according to any Form T1 is Ensartinib monohydrochloride hydrate.

According to any aspect or embodiment of the present disclosure, crystalline Form T1 of Ensartinib hydrochloride may be characterized by each of the above characteristics alone or by all possible combinations, e.g., by an X-ray powder diffraction pattern having peaks at 4.4, 8.2, 8.7, 11.7 and 13.3 degrees 2-theta±0.2 degrees 2-theta, a solid state ¹³C NMR spectrum as depicted in FIG. 10.

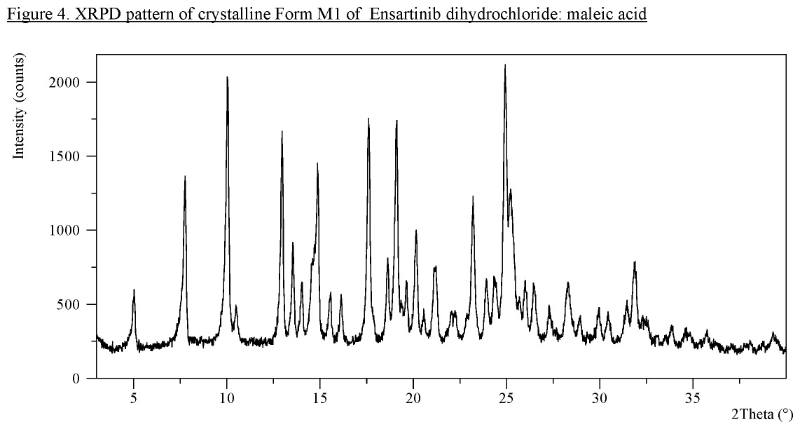

The present disclosure includes a crystalline polymorph of Ensartinib dihydrochloride: maleic acid, designated Form M1. The crystalline Form M1 of Ensartinib dihydrochloride: maleic acid may be characterized by data selected from one or more of the following: an X-ray powder diffraction pattern substantially as depicted in FIG. 4; an X-ray powder diffraction pattern having peaks at 7.8, 13.0, 13.6, 17.6 and 24.9 degrees 2-theta±0.2 degrees 2-theta; and combinations of these data.

According to any aspect or embodiment of the present disclosure, crystalline Form M1 of Ensartinib dihydrochloride: maleic acid may be further characterized by: an X-ray powder diffraction pattern having peaks at 7.8, 13.0, 13.6, 17.6 and 24.9 degrees 2-theta±0.2 degrees 2-theta, and also having any one, two, three, four or five additional peaks selected from 10.0, 14.0, 19.1, 20.2 and 23.2 degrees 2-theta±0.2 degrees 2-theta; or an X-ray powder diffraction pattern having peaks at 7.8, 10.0, 13.0, 13.6, 14.0, 17.6, 19.1, 20.2 and 23.2 and 24.9 degrees 2-theta 0.2 degrees 2-theta.

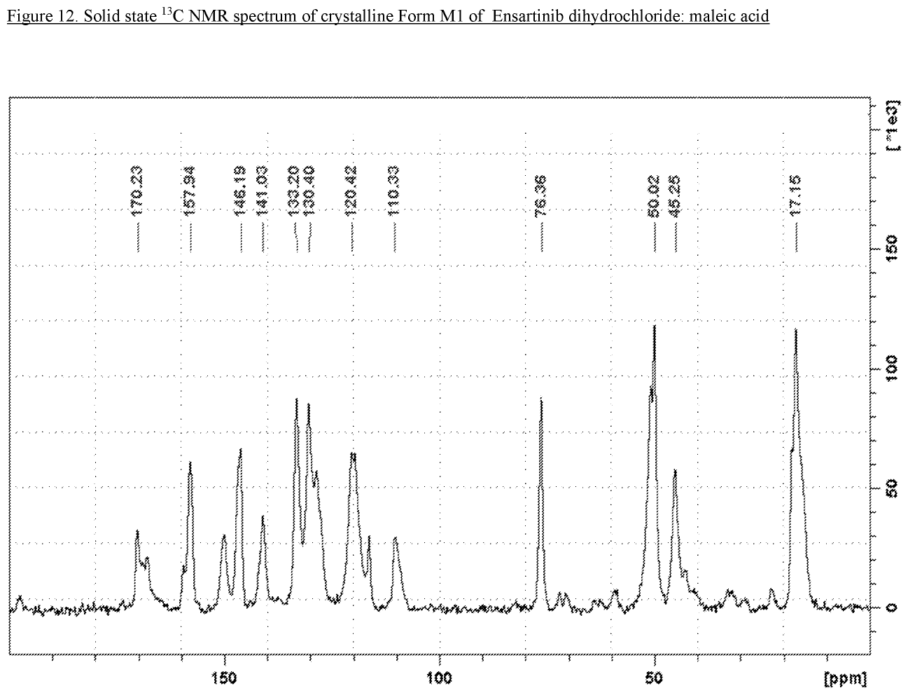

Alternatively, or additionally, according to any aspect or embodiment of the present disclosure, crystalline Form M1 of Ensartinib dihydrochloride: maleic acid may be characterized by data selected from one or more of the following: a solid state ¹³C NMR having peaks at 170.2, 157.9, 141.0, 130.4, 110.3±0.2 ppm; or a solid state ¹³C NMR spectrum having chemical shift differences between a reference peak at 76.4±0.2 ppm of: 93.8, 81.5, 64.6, 54.0, 33.9±0.1 ppm respectively; or by a solid state ¹³C NMR given in FIG. 12; and combinations of these data.

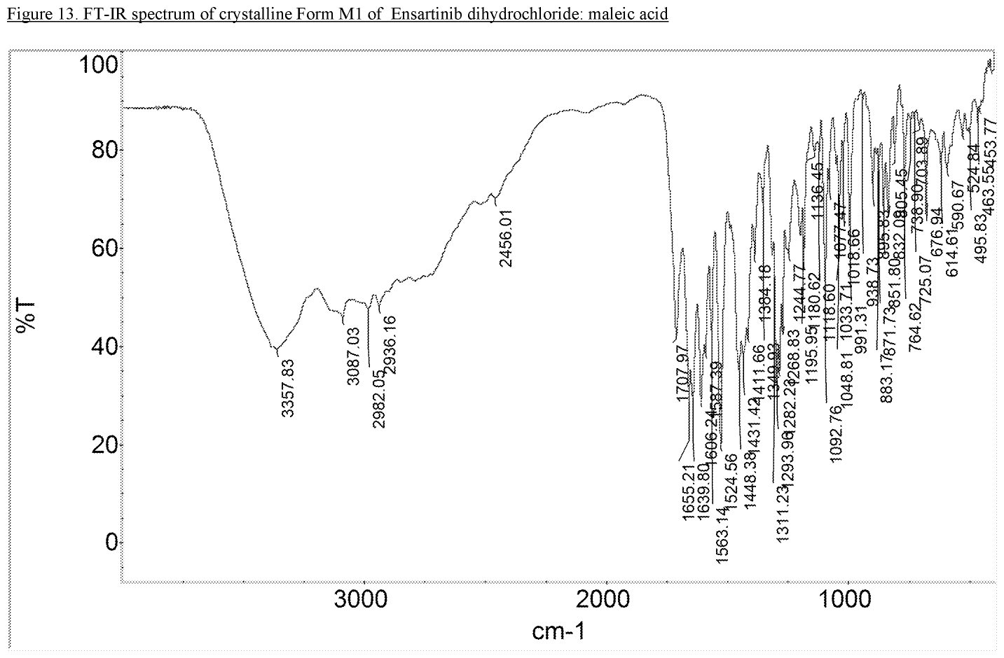

Crystalline Form M1 of Ensartinib dihydrochloride according to any aspect or embodiment of the present disclosure may be further characterized by a FT-IR spectrum substantially as depicted in FIG. 13.

According to any aspect or embodiment of the present disclosure, crystalline Form M1 of Ensartinib dihydrochloride: maleic acid may be characterized by each of the above characteristics alone or by all possible combinations, e.g., by an X-ray powder diffraction pattern having peaks at 7.8, 13.0, 13.6, 17.6 and 24.9 degrees 2-theta±0.2 degrees 2-theta, a solid state ¹³C NMR spectrum as depicted in FIG. 12.

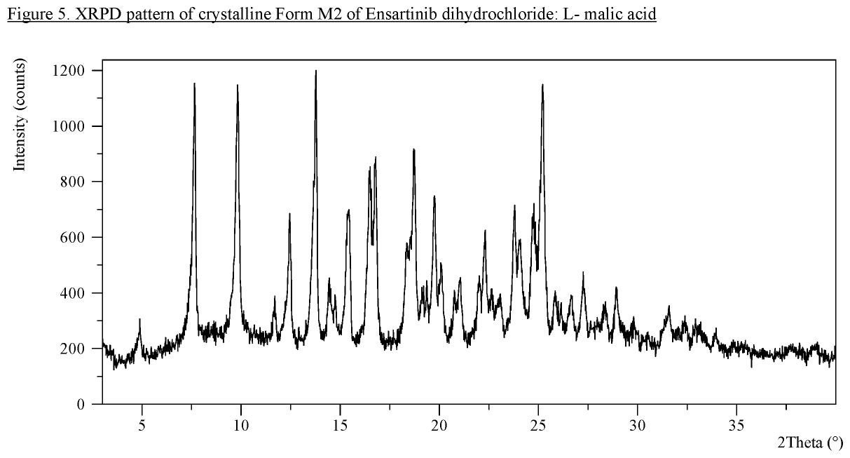

In another aspect, the current invention comprises a crystalline polymorph of Ensartinib dihydrochloride: L-malic acid, designated Form M2. The crystalline Form M2 of Ensartinib dihydrochloride: L-malic acid according to any aspect or embodiment of the present disclosure may be characterized by data selected from one or more of the following: an X-ray powder diffraction pattern substantially as depicted in FIG. 5; an X-ray powder diffraction pattern having peaks at 8.7, 12.4, 13.8, 16.8, 18.8 and 27.2 degrees 2-theta±0.2 degrees 2-theta; and combinations of these data.

According to any aspect or embodiment of the present disclosure, crystalline Form M2 of Ensartinib dihydrochloride: L-malic acid may be further characterized by: an X-ray powder diffraction pattern having peaks at 8.7, 12.4, 13.8, 16.8, 18.8 and 27.2 degrees 2-theta 0.2 degrees 2-theta, and also having any one, two, three, four or five additional peaks selected from 9.8, 16.5, 20.1, 22.3 and 25.2 degrees 2-theta±0.2 degrees 2-theta; or an X-ray powder diffraction pattern having peaks at 8.7, 9.8, 12.4, 13.8, 16.5, 16.8, 18.8, 20.1, 22.3, 25.2, and 27.2 degrees 2-theta±0.2 degrees 2-theta.

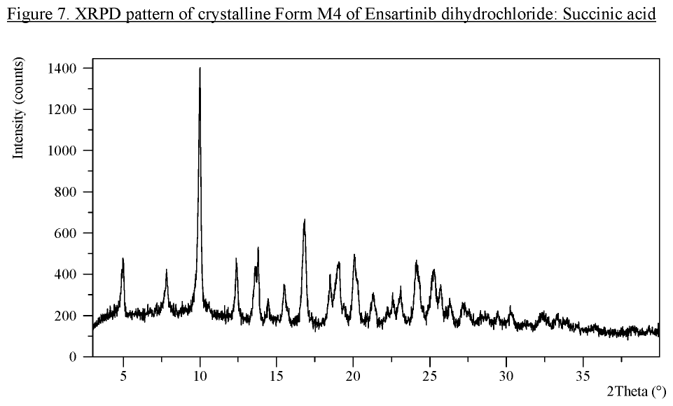

In a further embodiment, the present disclosure includes a crystalline polymorph of Ensartinib dihydrochloride: succinic acid, designated Form M4. The crystalline Form M4 of Ensartinib dihydrochloride: succinic acid may be characterized by data selected from one or more of the following: an X-ray powder diffraction pattern substantially as depicted in FIG. 7; an X-ray powder diffraction pattern having peaks at 8.0, 12.3, 15.5, 19.0 and 24.2 degrees 2-theta±0.2 degrees 2-theta; and combinations of these data.

According to any aspect or embodiment of the present disclosure, crystalline Form M4 of Ensartinib dihydrochloride: succinic acid may be further characterized by: an X-ray powder diffraction pattern having peaks at 8.0, 12.3, 15.5, 19.0 and 24.2 degrees 2-theta±0.2 degrees 2-theta, and also having any one, two, three, four or five additional peaks selected from 9.9, 13.5, 13.8, 16.8 and 21.4 degrees 2-theta±0.2 degrees 2-theta; or an X-ray powder diffraction pattern having peaks at 8.0, 9.9, 12.3, 13.5, 13.8, 15.5, 16.8, 19.0, 21.4, 24.2 degrees 2-theta±0.2 degrees 2-theta.

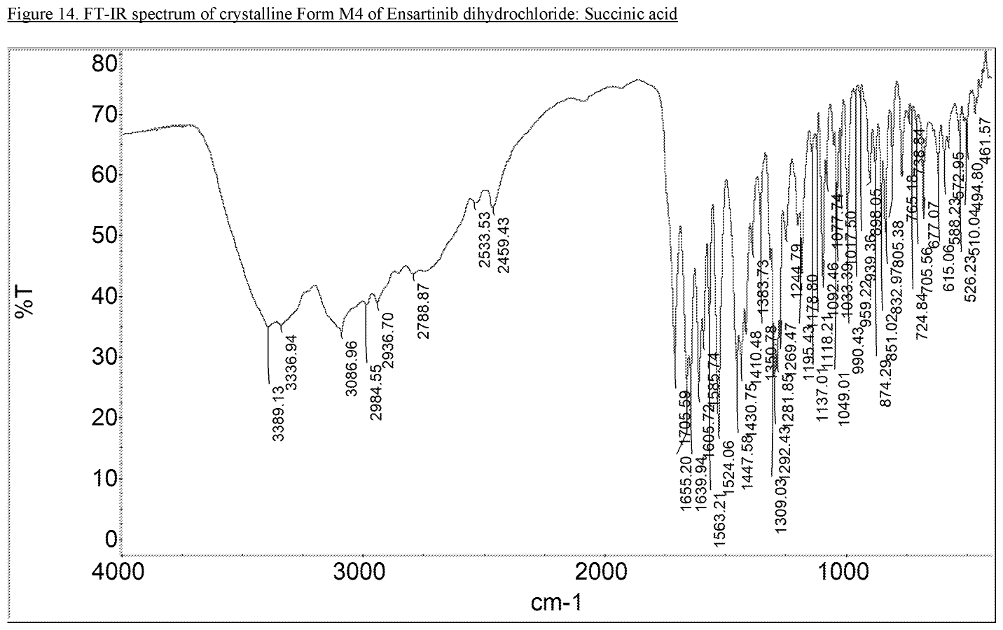

Crystalline Form M4 of Ensartinib hydrochloride according to any aspect or embodiment of the present disclosure may be further characterized by a FT-IR spectrum substantially as depicted in FIG. 14.

According to any aspect or embodiment of the present disclosure, the molar ratio between Ensartinib dihydrochloride and succinic acid is 2:1.

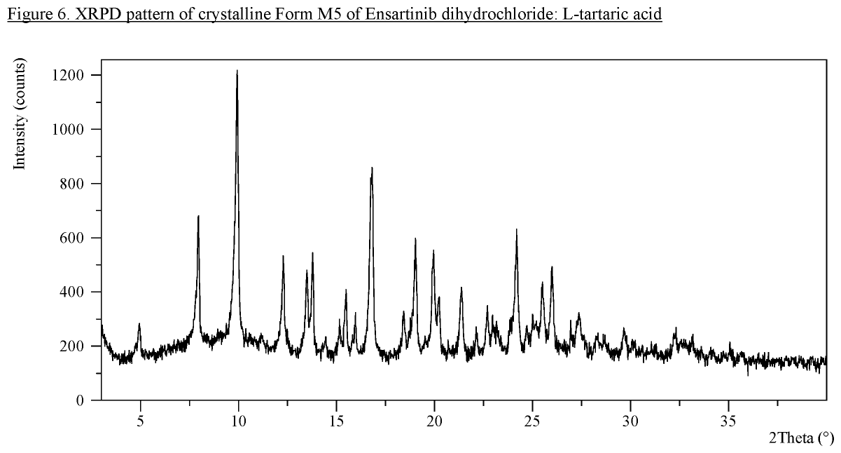

The present disclosure also includes a crystalline polymorph of Ensartinib dihydrochloride: L-tartaric acid, designated Form M5. The crystalline Form M5 of Ensartinib dihydrochloride: L-tartaric acid may be characterized by data selected from one or more of the following: an X-ray powder diffraction pattern substantially as depicted in FIG. 6; an X-ray powder diffraction pattern having peaks at 8.0, 12.3, 19.0, 24.2 and 26.0 degrees 2-theta±0.2 degrees 2-theta; and combinations of these data.

According to any aspect or embodiment of the present disclosure, crystalline Form M5 of Ensartinib dihydrochloride: L-tartaric acid may be further characterized by: an X-ray powder diffraction pattern having peaks at 8.0, 12.3, 19.0, 24.2 and 26.0 degrees 2-theta±0.2 degrees 2-theta, and also having any one, two, three, four or five additional peaks selected from 9.9, 13.5, 13.8, 16.8 and 21.4 degrees 2-theta±0.2 degrees 2-theta; or an X-ray powder diffraction pattern having peaks at 8.0, 9.9, 12.3, 13.5, 13.8, 16.8, 19.0, 21.4, 24.2 and 26.0 degrees 2-theta±0.2 degrees 2-theta.

According to any aspect or embodiment of the present disclosure, crystalline Form M5 of Ensartinib dihydrochloride: L-tartaric acid may be characterized by data selected from one or more of the following: an X-ray powder diffraction pattern substantially as depicted in FIG. 6; an X-ray powder diffraction pattern having peaks at 8.0, 12.3, 15.5, 19.0 and 24.2 degrees 2-theta±0.2 degrees 2-theta; and combinations of these data.

According to any aspect or embodiment of the present disclosure, crystalline Form M5 of Ensartinib dihydrochloride: L-tartaric acid may be further characterized by: an X-ray powder diffraction pattern having peaks at 8.0, 12.3, 15.5, 19.0 and 24.2 degrees 2-theta±0.2 degrees 2-theta, and also having any one, two, three, four or five additional peaks selected from 9.9, 13.5, 13.8, 16.8 and 21.4 degrees 2-theta±0.2 degrees 2-theta; or an X-ray powder diffraction pattern having peaks at 8.0, 9.9, 12.3, 13.5, 13.8, 15.5, 16.8, 19.0, 21.4, and 24.2 degrees 2-theta±0.2 degrees 2-theta.

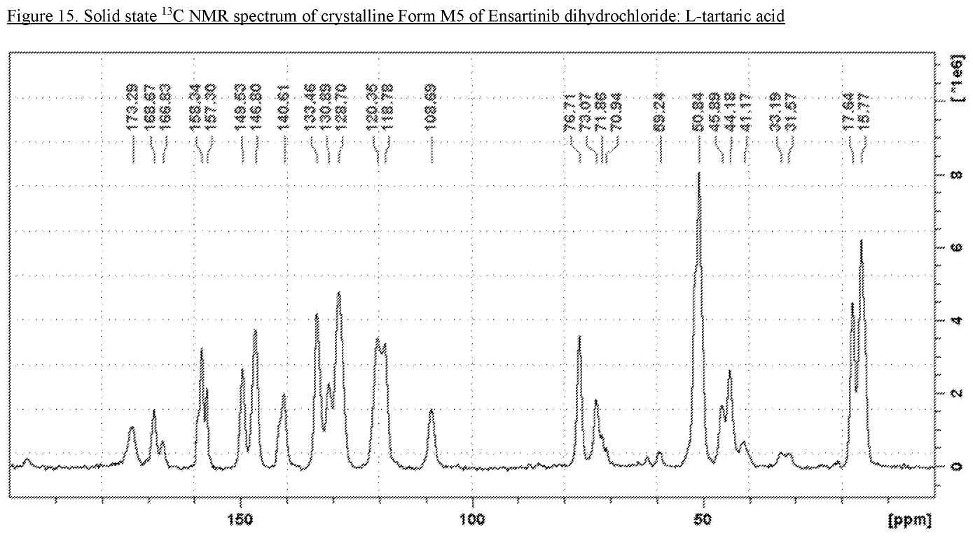

Alternatively, or additionally, according to any aspect or embodiment of the present disclosure, crystalline Form M5 of Ensartinib dihydrochloride: L-tartaric acid may be characterized by data selected from one or more of the following: a solid state ¹³C NMR having peaks at 173.3, 149.5, 146.8, 140.6, 133.5±0.2 ppm; or a solid state ¹³C NMR spectrum having chemical shift differences between a reference peak at 76.7±0.2 ppm of: 96.6, 72.8, 70.1, 63.9, 56.8±0.1 ppm respectively; or by a solid state ¹³C NMR substantially as depicted in FIG. 15; and combinations of these data.

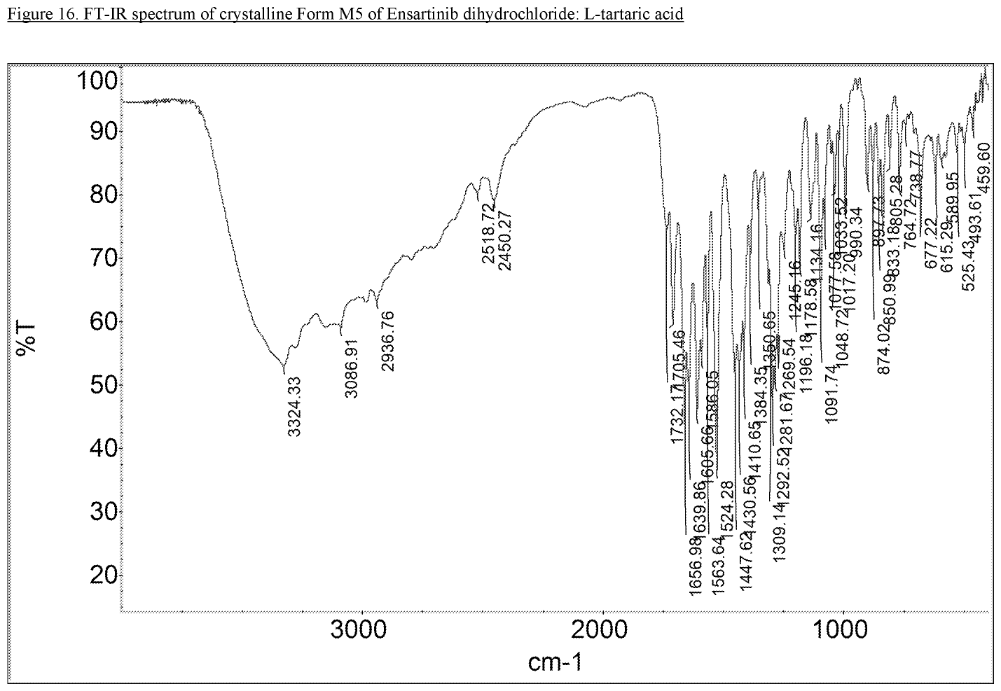

Crystalline Form M5 of Ensartinib dihydrochloride: L-tartaric acid according to any aspect or embodiment described herein, may be further characterized by a FT-IR spectrum substantially as depicted in FIG. 16.

According to any aspect or embodiment of the present disclosure, the molar ratio between Ensartinib dihydrochloride and L-tartaric acid is 2:1.

According to any aspect or embodiment of the present disclosure, crystalline Form M5 of Ensartinib dihydrochloride: L-tartaric acid may be characterized by each of the above characteristics alone or by all possible combinations, for example, an X-ray powder diffraction pattern having peaks at 8.0, 12.3, 15.5, 19.0 and 24.2 degrees 2-theta±0.2 degrees 2-theta and/or a FT-IR spectrum substantially as depicted in FIG. 16.

Crystalline forms M1, M2, M4 and M5 as described in any embodiment may be co-crystals of Ensartinib dihydrochloride with the respective acid.

According to any aspect or embodiment of the present disclosure, any of the solid state forms of Ensartinib, Ensartinib salts or co-crystals thereof described herein may be polymorphically pure or may be substantially free of any other solid state forms of the subject Ensartinib, Ensartinib salts or co-crystals thereof respectively. According to any aspect or embodiment of the present disclosure, any of the solid state forms of Ensartinib, Ensartinib salts or co-crystals thereof described herein may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of Ensartinib, Ensartinib salts or co-crystals thereof respectively, preferably as measured by XRPD. Thus, any of the disclosed crystalline forms of Ensartinib or Ensartinib salts and their co-crystals described herein may be substantially free of any other solid state forms of Ensartinib, Ensartinib salts or co-crystals thereof respectively, and may contain greater than about 80% (w/w), greater than about 90% (w/w), greater than about 95% (w/w), greater than about 98% (w/w), greater than about 99% (w/w), or about 100% of the subject solid state form of Ensartinib, Ensartinib salts or co-crystals thereof.

The above crystalline polymorphs and co-crystals can be used to prepare other crystalline polymorphs of Ensartinib, Ensartinib salts and their solid state forms.

The present disclosure encompasses a process for preparing other solid state forms of Ensartinib, Ensartinib salts and co-crystals and solid state forms thereof. The process includes preparing any one or a combination of the crystalline polymorphs or co-crystals of Ensartinib and/or of Ensartinib salts by the processes of the present disclosure, and converting these polymorphs to crystalline Ensartinib or Ensartinib salt. The conversion can be done, for example, by a process including acidifying any one or a combination of the above described solid state forms of Ensartinib to obtain the corresponding salt.

The present disclosure provides the above described crystalline polymorphs and co-crystals of Ensartinib and/or of Ensartinib salts for use in the preparation of pharmaceutical compositions comprising Ensartinib and/or crystalline polymorphs and co-crystals thereof.

The present disclosure also encompasses the use of crystalline polymorphs and co-crystals of Ensartinib and of Ensartinib salts of the present disclosure for the preparation of pharmaceutical compositions of Ensartinib or Ensartinib salts and/or crystalline polymorphs thereof.

The present disclosure includes processes for preparing the above mentioned pharmaceutical compositions. The processes include combining any one or a combination of the crystalline polymorphs or co-crystals of Ensartinib and/or of Ensartinib salts of the present disclosure with at least one pharmaceutically acceptable excipient.

Pharmaceutical combinations or formulations of the present disclosure contain any one or a combination of the solid state forms of Ensartinib and/or Ensartinib salts and co-crystals of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Ensartinib and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Ensartinib can be administered. Ensartinib may be formulated for administration to a mammal, in embodiments to a human, by injection. Ensartinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs and co-crystals of Ensartinib and of Ensartinib salts and the pharmaceutical compositions and/or formulations of Ensartinib and of Ensartinib salts of the present disclosure can be used as medicaments, in embodiments in the treatment of cancer, in particular non-small cell lung cancer (NSCLC), or in the treatment of melanoma with ALK alterations or aberrant ALK expression; or, in pediatric patients, for the treatment of: recurrent, refractory or advanced solid tumors, non-Hodgkin lymphoma, or histiocytic disorders with ALK or ROS1 genomic alterations. Preferably, the crystalline polymorphs and co-crystals of Ensartinib and of Ensartinib salts and the pharmaceutical compositions and/or formulations of Ensartinib and of Ensartinib salts of the present disclosure may be used in the treatment of cancer, in particular non-small cell lung cancer (NSCLC).

The present disclosure also provides methods of treating cancer, or for the treatment of melanoma with ALK alterations or aberrant ALK expression; or, in pediatric patients, for the treatment of: recurrent, refractory or advanced solid tumors, non-Hodgkin lymphoma, or histiocytic disorders with ALK or ROS1 genomic alterations; and preferably for the treatment of cancer, in particular non-small cell lung cancer (NSCLC), by administering a therapeutically effective amount of any one or a combination of the crystalline polymorphs or co-crystals of Ensartinib and/or of Ensartinib salts of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 Å (Angstrom), X'Celerator (2.022° 2θ) detector. Scanning parameters: angle range: 3-40 deg., step size 0.0167, time per step 37 s, continuous scan. The described peak positions were determined without using silicon powder as an internal standard in an admixture with the sample measured.

¹³C Solid State Nuclear Magnetic Resonance (“ss-NMR” or ¹³C Solid State NMR) Method

Solid state NMR spectra was measured at 11.7 T using a Bruker Avance III HD 500 US/WB NMR spectrometer (Karlsruhe, Germany, 2013). The ¹³C CP/MAS NMR spectra employing cross-polarization were acquired using the standard pulse scheme at spinning frequency of 11 kHz. The recycle delay was 8 s and the cross-polarization contact time was 2 ms. The strength of spin-locking fields B₁(¹³C) expressed in frequency units ω1/2π=γB1 was 64 kHz.

The ¹³C NMR scale was referenced to α-glycine (176.03 ppm). Frictional heating of the spinning samples was offset by active cooling, and the temperature calibration was performed with Pb(NO₃)₂. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δν1/2 was less than 3.5 Hz at 250 ms of acquisition time).

### FT-IR Method

IR spectrum was recorded on Nicolet 6700 FT-IR spectrometer operating in the range 4000-400 cm⁻¹, equipped with KBr beamsplitter and DTGS detector. 16 scans were recorded at resolution of 4.0 cm⁻¹. Sample was prepared as KBr pellet. Empty sample compartment was used for background spectrum acquisition. Measurements were taken at 293 K±3K.

### EXAMPLES

### Preparation of Starting Materials

Ensartinib can be prepared according to methods known from the literature, for example U.S. Pat. No. 9,126,947 (WO 2012/048259).

Ensartinib dihydrochloride (form A or amorphous or form B) can be prepared according to U.S. Patent Publication No. 2019/0135792 (WO 2017/206924).

Generally, amorphous Ensartinib can be prepared by grinding or by solvent-drop grinding, or by fast evaporation from a solution.

### Preparation of Amorphous Ensartinib Dihydrochloride

Ensartinib dihydrochloride (2×450 mg, Form B) was added to ZrO2 jars with seven ZrO2 balls (φ=9 mm). The sample was grinded on a rotational ball mill (600 rpm, 60 min). Solid was analyzed by XRPD, amorphous material was obtained.

Alternatively, amorphous Ensartinib dihydrochloride can be prepared as follows:

Ensartinib dihydrochloride (form A, 15 mg) was dissolved in a solvent mixture DCM/MeOH (1:1, 0.8 mL). The obtained solution was placed on a rota vapour, and the solvent was fast evaporated. The obtained solid was analyzed by XRPD. Amorphous Ensartinib dihydrochloride was obtained.

### Preparation of Crystalline Form a of Ensartinib Dihydrochloride

### Procedure 1:

Ensartinib dihydrochloride (500 mg, form B) was charged into a round-bottom flask with diisopropyl ether (14 ml) and a suspension was formed. Methanol (14 ml) was added and the obtained suspension was stirred at room temperature for 5 days. The obtained solid was isolated by vacuum filtration and dried on air. The sample was dried on oven by heating to a temperature of about 160° C. for 30 minutes. The solid was analyzed by XRPD, Form A was obtained.

### Procedure 2:

Ensartinib dihydrochloride (5000 mg, form B) was charged into a round-bottom flask with methanol (75 mL) and was heated to the boiling point (65° C.). The resulting solution was cooled to a temperature of about 25° C. and was stirred for 5 days. The solid was isolated by vacuum filtration and dried on air. A sample of the solid was dried in vacuum (50° C.) for 4 hours. The dried sample was analyzed by XRPD. Form A was obtained.

### Procedure 3:

Ensartinib monohydrochloride (2234 mg, form T1) was charged into a round-bottom flask with methanol (72 mL) and heated to the boiling point (65° C.) and a solution formed. Concentrated HCl (0.7 ml) was added and the solution cooled to 25° C., and then further cooled in an ice bath for 30 minutes. The solid was isolated by vacuum filtration and dried for 4 hours at 60° C. A sample was analyzed by XRPD, Form A was obtained.

### Example 1: Preparation of Ensartinib Form 1

Ensartinib (30 mg) was dissolved in acetone (1.0 mL) at 25° C. Solution was left in an open vial for solvent to evaporate (at room temperature). Obtained solid was isolated by vacuum filtration and dried in air for 10 minutes. The obtained solid was analyzed by XRPD. Crystalline Ensartinib Form 1 was obtained. XRPD pattern is given in FIG. 1.

### Example 2: Preparation of Ensartinib Form 2

Ensartinib (30 mg) was dissolved in pentane-3-one (1.0 mL) at 50° C. Solution was left in an open vial for solvent to evaporate (at room temperature). Obtained solid was isolated by vacuum filtration and dried in air for 10 minutes. The obtained solid was analyzed by XRPD. Crystalline Ensartinib Form 2 was obtained. XRPD pattern is given in FIG. 2.

### Example 3: Preparation of Ensartinib Hydrochloride Salt from T1

Ensartinib (5.0 grams) was dissolved in acetone (100 mL) at boiling point. Solution was cooled to 25° C. and to the stirred solution was added hydrochloric acid (conc., 0.84 mL, 1.1 eq.). The resulting suspension was stirred for 30 minutes at 25° C. The obtained solid was isolated by vacuum filtration and dried by stream of nitrogen for 15 minutes. The obtained solid was analyzed by XRPD; Crystalline Ensartinib hydrochloride salt Form T1 was obtained. XRPD pattern is given in FIG. 3.

### Example 4: Preparation of Ensartinib Dihydrochloride: Maleic Acid Form M1

Ensartinib dihydrochloride (form A, 500 mg) and maleic acid (105 mg) were suspended in ethyl acetate (10 mL) at temperature of 50° C. for 4 days. Solid was isolated by vacuum filtration over black ribbon filter paper. The obtained solid was analyzed by XRPD; Crystalline Form M1 of Ensartinib dihydrochloride: maleic acid was obtained. XRPD pattern is given in FIG. 4.

### Example 5: Preparation of Ensartinib Dihydrochloride: Maleic Acid Form M1

Ensartinib dihydrochloride (amorphous, 100 mg) and maleic acid (21 mg) were suspended in ethyl acetate (3 mL) at temperature of 50° C. for 4 days. Solid was isolated by vacuum filtration over black ribbon filter paper. The obtained solid was analyzed by XRPD; Crystalline Form M1 of Ensartinib dihydrochloride: maleic acid was obtained.

### Example 6: Preparation of Ensartinib Dihydrochloride: Maleic Acid Form M1

Ensartinib dihydrochloride (form A, 500 mg) and maleic acid (105 mg) were suspended in ethyl acetate (10 mL) at temperature of 50° C. for 4 days. Suspension was allowed to cool to room temperature (about 1 hour) and solid was isolated by vacuum filtration over black ribbon filter paper. The obtained solid was analyzed by XRPD; Crystalline Form M1 of Ensartinib dihydrochloride: maleic acid was obtained.

### Example 7: Preparation of Ensartinib Dihydrochloride: L-Malic Acid Form M2

Ensartinib dihydrochloride (form A, 500 mg) and L-malic acid (214 mg) were suspended in isopropyl acetate (10 mL) at temperature of 50° C. for 4 days. Solid was isolated by vacuum filtration over black ribbon filter paper. The obtained solid was analyzed by XRPD; Crystalline Form M2 of Ensartinib dihydrochloride: L-malic acid was obtained. XRPD pattern is given in FIG. 5.

### Example 8: Preparation of Ensartinib Dihydrochloride: L-Malic Acid Form M2

Ensartinib dihydrochloride (amorphous, 100 mg) and L-malic acid (42 mg) were suspended in isopropyl acetate (3 mL) at temperature of 50° C. for 4 days. Solid was isolated by vacuum filtration over black ribbon filter paper. The obtained solid was analyzed by XRPD; Crystalline Form M2 of Ensartinib dihydrochloride: L-malic acid was obtained.

### Example 9: Preparation of Ensartinib Dihydrochloride: L-Malic Acid Form M2

Ensartinib dihydrochloride (form A, 500 mg) and L-malic acid (214 mg) were suspended in isopropyl acetate (10 mL) at temperature of 50° C. for 4 days. Suspension was allowed to cool to room temperature (about 1 hour) and solid was isolated by vacuum filtration over black ribbon filter paper. The obtained solid was analyzed by XRPD; Crystalline Form M2 of Ensartinib dihydrochloride: L-malic acid was obtained.

### Example 10: Preparation of Ensartinib Dihydrochloride: L-Tartaric Acid Form M5

Ensartinib dihydrochloride (form A, 200 mg) was dissolved in 2.5 mL of methanol/acetonitrile (3:1) at 60° C. L-tartaric acid (96 mg) was added to the solution at 60° C. Obtained solution was cooled to room temperature (25° C.). Pre-cooled (0-5° C.) 2-propanol (5 ml) was added dropwise to the solution. Crystallization occurred. Suspension was stirred for 7 days at room temperature (25° C.). Solid was isolated by vacuum filtration over black ribbon filter paper. The obtained solid was analyzed by XRPD; Crystalline Form M5 of Ensartinib dihydrochloride: L-tartaric acid was obtained. XRPD pattern is given in FIG. 6.

### Example 11: Preparation of Ensartinib Dihydrochloride: L-Tartaric Acid Form M5

Ensartinib dihydrochloride (200 mg) was dissolved in a mixture of MeOH and ACN (3:1, total 2.5 mL) at a temperature of 60° C. L-tartaric acid (96 mg) was added to the solution at 60° C. The obtained solution was cooled down to room temperature (25° C.). Cooled 2-propanol (5 mL, pre-cooled to 0-4° C.) was added dropwise to the solution and crystallization occurred. Additional 50 mg of L-tartaric acid was added and the obtained suspension was stirred for 3 days at room temperature (25° C.). The obtained solid was isolated by vacuum filtration over black ribbon filter paper. The obtained solid was analyzed by XRPD. Crystalline form of Ensartinib dihydrochloride and L-tartaric acid Form M5 was obtained.

### Example 12: Preparation of Ensartinib Dihydrochloride: Succinic Acid Form M4

Ensartinib dihydrochloride (form A, 1.15 grams) and succinic acid (230 grams) were suspended in 2-propanol (10 mL) at temperature of 50° C. for 4 days. Suspension was allowed to cool to room temperature (about 1 hour) and solid was isolated by vacuum filtration over black ribbon filter paper. The obtained solid was analyzed by XRPD; Crystalline Form M4 of Ensartinib dihydrochloride: succinic acid was obtained. XRPD pattern is given in FIG. 7.

1. Crystalline Form M5 of Ensartinib dihydrochloride: L-tartaric acid characterized by data selected from one or more of the following:
a) an X-ray powder diffraction pattern substantially as depicted in FIG. 6;
b) an X-ray powder diffraction pattern having peaks at 8.0, 12.3, 19.0, 24.2 and 26.0 degrees 2-theta±0.2 degrees 2-theta;
c) an X-ray powder diffraction pattern having peaks at 8.0, 12.3, 15.5, 19.0 and 24.2 degrees 2-theta±0.2 degrees 2-theta;
d. a solid state ¹³C NMR spectrum having peaks at 173.3, 149.5, 146.8, 140.6, and 133.5±0.2 ppm;
e) a solid state ¹³C NMR spectrum having chemical shift differences between a reference peak at 96.6, 72.8, 70.1, 63.9, and 56.8±0.1 ppm respectively;
f. a solid state ¹³C NMR substantially as depicted in FIG. 15; and
g. any combination of (a)-(f).

2. Crystalline Form M5 of Ensartinib dihydrochloride: L-tartaric acid according to claim 1, characterized by an X-ray powder diffraction pattern having peaks at 8.0, 12.3, 19.0, 24.2 and 26.0 degrees 2-theta±0.2 degrees 2-theta, and also having any one, two, three, four or five additional peaks selected from 9.9, 13.5, 13.8, 16.8 and 21.4 degrees 2-theta±0.2 degrees 2-theta; or an X-ray powder diffraction pattern having peaks at 8.0, 9.9, 12.3, 13.5, 13.8, 16.8, 19.0, 21.4, 24.2, and 26.0 degrees 2-theta±0.2 degrees 2-theta.

3. Crystalline Form M5 of Ensartinib dihydrochloride: L-tartaric acid according to claim 1, characterized by an X-ray powder diffraction pattern having peaks at 8.0, 12.3, 15.5, 19.0 and 24.2 degrees 2-theta±0.2 degrees 2-theta, and also having any one, two, three, four or five additional peaks selected from 9.9, 13.5, 13.8, 16.8 and 21.4 degrees 2-theta±0.2 degrees 2-theta; or an X-ray powder diffraction pattern having peaks at 8.0, 9.9, 12.3, 13.5, 13.8, 15.5, 16.8, 19.0, 21.4, and 24.2 degrees 2-theta±0.2 degrees 2-theta.

4. Crystalline Form M5 of Ensartinib dihydrochloride: L-tartaric acid according to claim 1, which is further characterized by FT-IR spectrum substantially as depicted in FIG. 16.

5. Crystalline Form M5 of Ensartinib dihydrochloride: L-tartaric acid according to claim 1, which contains no more than about 20% of any other crystalline forms of Ensartinib dihydrochloride: L-tartaric acid; and/or no more than about 20% of amorphous Ensartinib dihydrochloride: L-tartaric acid.

6. Crystalline Form T1 of Ensartinib hydrochloride salt characterized by data selected from one or more of the following:
a) an X-ray powder diffraction pattern substantially as depicted in FIG. 3;
b) an X-ray powder diffraction pattern having peaks at 4.4, 8.2, 8.7, 11.7, and 13.3 degrees 2-theta±0.2 degrees 2-theta;
c) a solid state ¹³C NMR having peaks at 167.9, 142.3, 130.7, 118.3, and 105.8±0.2 ppm;
d) a solid state ¹³C NMR spectrum having chemical shift differences between a reference peak at 77.3±0.2 ppm of 90.6, 65.0, 53.4, 41.0, and 28.5±0.1 ppm respectively;
e) a solid state ¹³C NMR spectrum substantially as depicted in FIG. 10; and
f) combinations of these data.

7. Crystalline Form T1 of Ensartinib hydrochloride salt according to claim 6, characterized by an X-ray powder diffraction pattern having peaks at 4.4, 8.2, 8.7, 11.7, and 13.3 degrees 2-theta±0.2 degrees 2-theta, and also having any one, two, three, four or five additional peaks selected from 14.0, 15.9, 17.6, 18.2, and 19.8 degrees 2-theta±0.2 degrees 2-theta; or an X-ray powder diffraction pattern having peaks at 4.4, 8.2, 8.7, 11.7, 13.3, 14.0, 15.9, 17.6, 18.2, and 19.8 degrees 2-theta±0.2 degrees 2-theta.

8. Crystalline Form T1 of Ensartinib hydrochloride salt according to claim 6, which is further characterized by a FT-IR spectrum substantially as depicted in FIG. 11.

9. Crystalline Form T1 of Ensartinib hydrochloride salt according to claim 6, which is a mono-hydrochloride salt.

10. Crystalline Form T1 of Ensartinib hydrochloride salt according to claim 6, which is a hydrate form.

11. Crystalline Form T1 of Ensartinib hydrochloride salt according to claim 6, which contains no more than about 20% of any other crystalline forms of hydrochloride salt; and/or no more than about 20% of amorphous Ensartinib hydrochloride salt.

12. A pharmaceutical composition comprising a crystalline form according to claim 1.

13. A pharmaceutical formulation comprising a crystalline form according to claim 1 and at least one pharmaceutically acceptable excipient.

14. A process for preparing a pharmaceutical formulation comprising combining a crystalline form according to claim 1 with at least one pharmaceutically acceptable excipient.

15. (canceled)

16. A medicament comprising the crystalline form according to claim 1.

17. (canceled)

18. A method of treating cancer, optionally for use in the treatment of non-small cell lung cancer (NSCLC), or in the treatment of melanoma with ALK alterations or aberrant ALK expression; or, in paediatric patients, in the treatment of recurrent, refractory or advanced solid tumors, non-Hodgkin lymphoma, or histiocytic disorders with ALK or ROS1 genomic alterations, comprising administering a therapeutically effective amount of a crystalline form according to claim 1 to a subject in need of the treatment.

19. (canceled)

20. (canceled)

21. (canceled)
